(19) United States
(12) Patent Application Publication
(10) Pub. No.: US 2008/0292716 A1
(43) Pub. Date: Nov. 27, 2008

(54) Electrolytic Water and Process for Production Thereof

(75) Inventors: Kokichi Hanaoka, Nagano (JP); Masako Morito, Tokyo (JP)

Correspondence Address: Bruce S. Londa;Norris McLaughlin & Marcus, PA, 18th Floor, 875 Third Avenue, New York, NY, 10022, US

(73) Assignees: Kokichi Hanaoka, Nagano (JP); MIKUNI CORPORATION, Tokyo (JP)

(21) Appl. No.: 11/741,212

(22) Filed: Apr. 27, 2007

(30) Foreign Application Priority Data
Apr. 29, 2006 (JP) 2006-124793

(51) Int. Cl. A61K 33/00

(52) U.S. Cl. 424/600

(57) ABSTRACT

The present invention discloses a process for producing an electrolytic water, wherein a to-be-electrolyzed water containing less than 0.1 mM of a water-soluble inorganic salt, 0.05 to 0.5 mass % of a hydroquinone derivative and 1 to 50 mM of L-ascorbic acid or a derivative thereof is fed into a diaphragm-free electrolytic cell of continuous flow type at a flow rate of 5 to 3,000 l/min and electrolysis is conducted continuously at a current density of 0.003 to 0.1 A/cm². The process can produce an electrolytic water having a high skin-whitening effect owing to the reducing action of L-ascorbic acid and the melanin formation-suppressing effect of hydroquinone derivative.

### TECHNICAL FIELD

The present invention relates to an electrolytic water and a process for production thereof. More particularly, the present invention relates to an electrolytic water useful for skin care, obtained by electrolyzing an aqueous L-ascorbic acid solution containing a hydroquinone derivative, as well as to a process for producing such an electrolytic water.

### BACKGROUND ART

L-ascorbic acid is ionized in water by the dissociation of 2- and 3-position hydroxyl groups of the chemical structure. It is a well-known technique to apply a voltage to an aqueous L-ascorbic acid solution to conduct electrolysis thereof (reference is made to, for example, Japanese Patent No. 3602773).

When an aqueous L-ascorbic acid solution is electrolyzed, the hydrogen ion and the ascorbic acid ion both present as a result of the dissociation of ascorbic acid in water migrate to electrodes of opposite polarity, respectively. Simultaneously, the water as a solvent is electrolyzed; as a result, on the anode surface, oxidation takes place to generate oxygen and hydrogen ion and, on the cathode surface, reduction takes place to generate hydrogen and hydroxyl ion.

The hydrogen ion and the ascorbic acid ion both present as a result of the dissociation of ascorbic acid in water react, at respective electrodes, with the ions generated by electrolysis of water, to form electrolysis products. That is, the ascorbic acid ion reacts, on the cathode, with the hydrogen ion (H⁺) formed by the oxidation of water, to become ascorbic acid. On the anode, the hydrogen ion reacts with the hydroxyl ion formed by the reduction of water, to produce water. The oxygen generated on the cathode surface and the hydrogen generated on the anode surface remain in water as dissolved oxygen or dissolved hydrogen.

It is generally known that, in dissolution of a water-soluble substance in water (solvent), the dissolution characteristic of the water-soluble substance varies depending upon the conditions of the solvent. When the solvent is water, the interaction between water-soluble substance and water varies depending upon conditions such as temperature, pressure and the like, resulting in variation of the dissolution characteristic of the water-soluble substance.

An aqueous electrolyte solution has, under particular conditions, an enhanced entropy when electrolyzed and accordingly comes to have a higher energy than in its steady state. As a result, the function of water as a solvent is activated and the degree of dissociation of water per se tends to become higher. In the electrolytic water obtained by electrolysis of aqueous electrolyte solution, the dissolution degree of water is higher and thereby the water molecules surrounding the elute come to have a different conformation. According to some reports published, this difference (change) in conformation brings about a higher dissolution rate of electrolyte or a higher dissociation degree of substance of low dissolution degree.

When an aqueous ascorbic acid solution is electrolyzed, the water as a solvent is influenced by the electrolysis and activated. The water gives rise to a structural change, which brings about a higher dissociation degree of ascorbic acid and a higher solubility thereof. Also, there occurs a change in the interaction between water (solvent) and ascorbic acid (solute), which is considered to bring about a higher reactivity of ascorbic acid per se.

The present inventors paid attention to these actions in the electrolysis of aqueous ascorbic acid solution and made various studies in order to obtain an electrolytic water maintaining this ability. As a result, it was found that, when an aqueous solution containing only ascorbic acid of relatively low concentration is electrolyzed without using an inorganic electrolyte (e.g. a water-soluble metal salt) as an electrolysis aid, there can be obtained an anode side electrolytic water which maintains dismutation ability for superoxide radical; and a patent was acquired therefor (Japanese Patent No. 3602773).

### DISCLOSURE OF THE INVENTION

It has been confirmed that the above-mentioned electrolytic water obtained by electrolysis of aqueous ascorbic acid solution, when coated on skin, shows, owing to the characteristic change brought about by electrolysis, higher emulsification of the sebum present on skin and higher permeation of ascorbic acid into skin. Also, it has been made clear that the ascorbic acid, which has permeated into skin, acts as a strong reducing agent and the above electrolytic water, when coated on skin, shows a high skin-whitening effect owing to the reductive action of ascorbic acid per se.

Meanwhile, it is known that hydroquinone and its derivative have a suppressive effect for melanin formation and, when coated on skin, are effective for the amelioration of sunburn, blotch, dark spot, etc. Hydroquinone has a strong side effect and is used in medicines, while a hydroquinone derivative is low in side effect and used mainly in cosmetics. It has been made clear that, of various hydroquinone derivatives, hydroquinone glycosides are low in side effect, have a high affinity to living body, and are superior in prolonged suppressive effect for melanin formation. The hydroquinone glycosides include, for example, α-arbutin and β-arbutin (both are glucose adducts) having the following chemical structures.

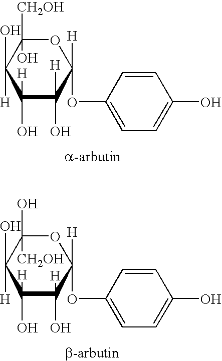

The hydroquinone glycoside is water-soluble but is not ionized sufficiently in water; therefore, when an aqueous solution thereof is electrolyzed, the hydroquinone glycoside per se does not take part in the electrolysis. An investigation by the present inventors revealed that, when a pure water containing only a water-soluble hydroquinone derivative (e.g. a-arbutin) is electrolyzed, there is substantially no electrolysis and there is no change in its suppressive effect for melanin formation.

The present inventors paid attention to the above feature of hydroquinone derivative and anticipated that there might be obtained an electrolytic water of high skin-whitening effect, useful for skin care, by adding a hydroquinone derivative to an electrolytic water obtained by electrolysis of aqueous ascorbic acid solution.

The aim of the present invention is to obtain an electrolytic water having a higher skin-whitening effect than heretofore, by electrolyzing an aqueous water containing both ascorbic acid and a hydroquinone glycoside and allowing the hydroquinone derivative (which has not substantially taken part in the electrolysis) to be present in the obtained electrolytic water wherein water has undergone a structure change.

The present inventors made various studies and, as a result, found that, by electrolyzing an aqueous water containing a hydroquinone derivative and ascorbic acid or its derivative, there can be obtained an electrolytic water showing a higher skin-whitening effect than conventional electrolytic waters obtained by electrolysis of aqueous ascorbic acid solution.

The present invention, which has achieved the above aim, is as follows.

[1] An electrolytic water containing less than 0.1 mM of a water-soluble inorganic salt, 0.05 to 0.5 mass % of a hydroquinone derivative and 1 to 50 mM of L-ascorbic acid or a derivative thereof.
[2] An electrolytic water according to [1], wherein the L-ascorbic acid derivative is sodium ascorbyl phosphate and/or magnesium ascorbyl phosphate.
[3] An electrolytic water according to [1], wherein the hydroquinone derivative is a-arbutin or B-arbutin.
[4] A process for producing an electrolytic water, which comprises electrolyzing a to-be-electrolyzed water containing less than 0.1 mM of a water-soluble inorganic salt, 0.05 to 0.5 mass % of a hydroquinone derivative and 1 to 50 mM of L-ascorbic acid or a derivative thereof and taking out the electrolytic water obtained.
[5] A process for producing an electrolytic water according to [4], wherein the L-ascorbic acid derivative is sodium ascorbyl phosphate and/or magnesium ascorbyl phosphate.
[6] A process for producing an electrolytic water according to [4], wherein the hydroquinone derivative is α-arbutin or β-arbutin.
[7] A process for producing an electrolytic water according to [4], wherein the electrolysis is conducted at a current density of 0.003 to 0.1 A/cm².
[8] A process for producing an electrolytic water according to [4], wherein the to-be-electrolyzed water containing less than 0.1 mM of a water-soluble inorganic salt, 0.05 to 0.5 mass % of a hydroquinone derivative and 1 to 50 mM of L-ascorbic acid or a derivative thereof is fed into a diaphragm-free electrolytic cell of continuous flow type at a flow rate of 5 to 3,000 l/min and the electrolysis is conducted continuously at a current density of 0.003 to 0.1 A/cm².

The electrolytic water of the present invention, which is obtained by electrolyzing a to-be-electrolyzed water containing an L-ascorbic acid derivative as an effective ingredient and also as an electrolysis aid and further containing a hydroquinone derivative, has a reducing action of L-ascorbic acid and a melanin formation-suppressing effect of hydroquinone derivative, and can be used as a cosmetic of very high skin-whitening effect. The ascorbic acid derivative and hydroquinone derivative contained in to-be-electrolyzed water are high in safety to human body and the electrolytic water of the present invention obtained by electrolysis of such a to-be-electrolyzed water is extremely high in safety as well.

The hydroquinone derivative per se does not substantially take part in electrolysis. Owing to the enhanced solubility brought about by the change in interaction between solvent and solute, taking place in electrolysis of to-be-electrolyzed water, the dissolved oxygen present in the electrolytic water produced, etc., the electrolytic water of the present invention shows high permeability when coated on skin.

Needless to say, the electrolytic water of the present invention has a suppressive effect for melanin formation which is not possessed by conventional electrolytic waters obtained by electrolysis of aqueous ascorbic acid solution. With the present electrolytic water, there can further be expected, when coated on skin, superiorities in skin smoothness, stick-free feeling, etc. and improvements in makeup, etc.

### BRIEF DESCRIPTION OF THE DRAWINGS

FIG. 1 is a schematic view showing an example of the electrolytic apparatus used in the process for producing an electrolytic water according to the present invention.

FIG. 2 is a schematic sectional view showing other example of the electrolytic apparatus used in the process for producing an electrolytic water according to the present invention.

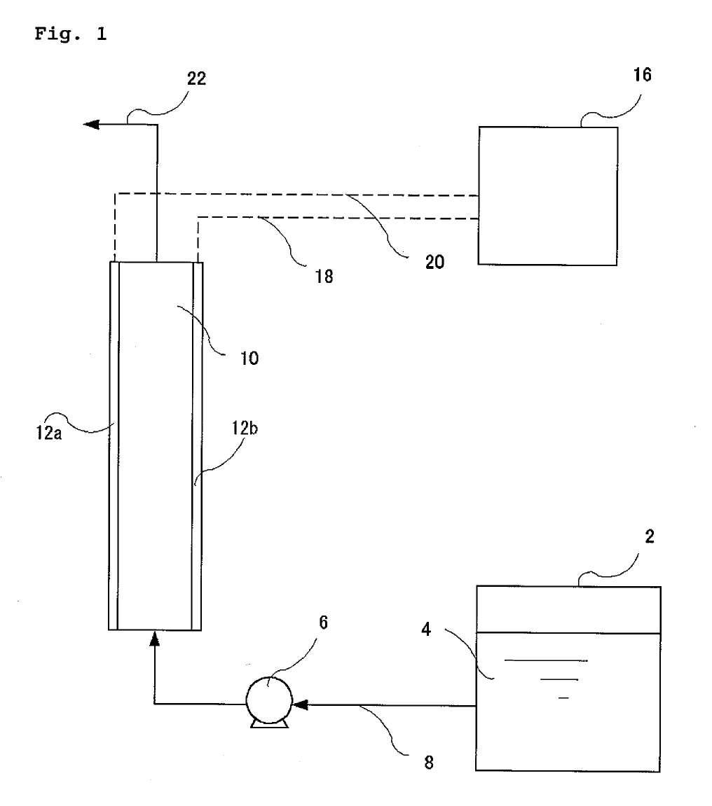

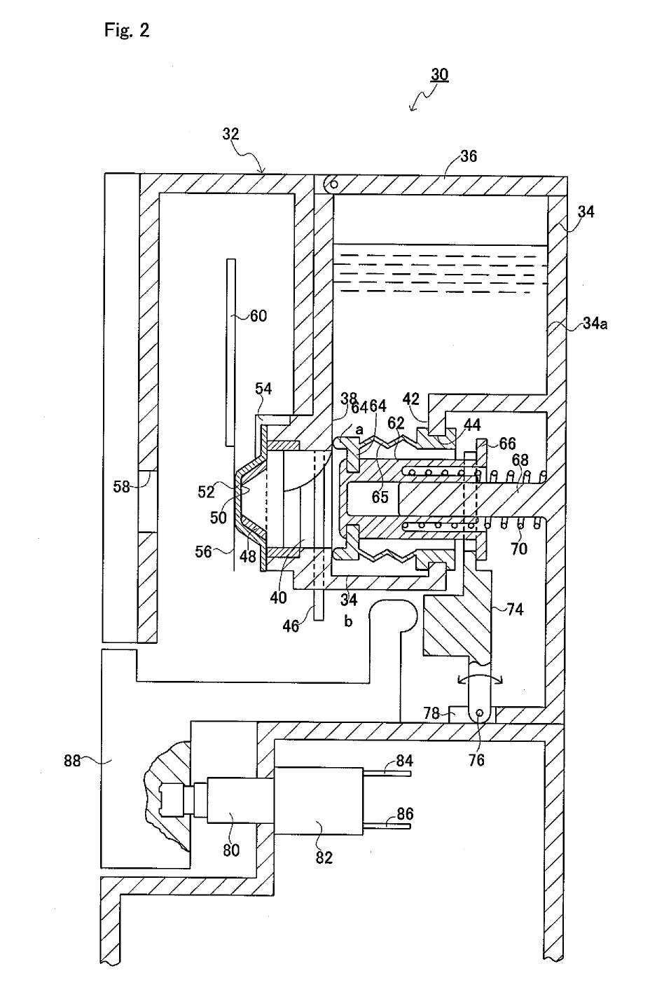

In FIG. 1 and FIGS. 2, 2 and 34 are each a tank for to-be-electrolyzed water; 4 is an aqueous electrolyte solution; 6 is a pump; 8 is a pipe for feeding of aqueous electrolyte solution; 10 is a diaphragm-free electrolytic cell; 12a, 12b and 46 are each an electrode; 16 is an electric source for electrolysis; 18, 20, 84 and 86 are each a wiring; 30 is an electrolytic apparatus; 32 is a casing; 38 is a front side wall; 40 is an electrolytic chamber; 42 is a back side wall; 44 is a port for plunger insertion; 50 is a feeding port; 52 is a solution-absorbing medium; 56 is a perforated plate; 58 is a spray port; 60 is a piezo trembler; 62 is a valve-driven plunger; 64 is a switching valve; 66 is a flange; 68 is a guide shaft; 70 is a compression coil spring; 74 is a rotary lever; and 88 is a push button.

### BEST MODE FOR CARRYING OUT THE INVENTION

In the electrolytic water of the present invention, the content of L-ascorbic acid or its derivative is 1 to 50 mM, preferably 2 to 30 mM. When the content of L-ascorbic acid or its derivative is less than 1 mM, the conductivity in electrolysis is low, making difficult the electrolysis. When the content is more than 50 mM, there is a sticky feeling when the electrolytic water obtained has been applied to skin, etc.

As the L-ascorbic acid derivative, there are alkali metal salts, alkaline earth metal salts, ammonium salt, etc. of L-ascorbic acid. There can be mentioned, for example, sodium ascorbyl phosphate and magnesium ascorbyl phosphate.

As the hydroquinone derivative compounded in the electrolytic water, there can be used known water-soluble derivatives compounded in cosmetics, etc. There is preferably used a hydroquinone glycoside because it is superior in prolonged suppressive effect for melanin formation and shows a low side effect. As the hydroquinone glycoside, there can be mentioned, for example, α-arbutin and β-arbutin (which are both glucose adducts).

In the electrolytic water, the content of hydroquinone derivative is 0.05 to 0.5 mass %, preferably 0.08 to 0.3 mass %. When the content is less than 0.05 mass %, the suppressive effect for melanin formation is not sufficient and there is no sufficient wet feeling when the electrolytic water has been coated on skin. When the content is more than 0.5 mass %, there is strong tight feeling, making inferior the feeling when the electrolytic water has been coated on skin.

The electrolytic water of the present invention may contain other components in amounts which do not impair the effects of the present invention. As specific examples of such other components, there can be mentioned organic acids (e.g. citric acid) compounded for the promoted osmosis into skin or for the control of pH, and amino acids for higher humectation effect.

Next, the process for producing an electrolytic water according to the present invention is described with reference to FIG. 1.

FIG. 1 is a schematic view showing an example of the electrolytic apparatus used in the process for producing an electrolytic water according to the present invention.

In FIG. 1, 2 is a tank for to-be-electrolyzed water, in which an aqueous electrolyte water (a to-be-electrolyzed water) 4 is stored.

The to-be-electrolyzed water 4 contains 1 to 50 mM, preferably 2 to 30 mM of L-ascorbic acid or its derivative and 0.05 to 0.5 mass %, preferably 0.08 to 0.3 mass % of a hydroquinone derivative.

Desirably, the to-be-electrolyzed water 4 substantially contains, as an electrolysis aid, an electrolyte (e.g. a water-soluble inorganic salt) other than L-ascorbic acid or its derivative. The content of the water-soluble inorganic electrolyte is preferably less than 0.1 mM, particularly preferably 0.02 mM or less in terms of the total of individual water-soluble inorganic electrolytes.

As the method for preparing the to-be-electrolyzed water 4, there is, for example, a method of dissolving L-ascorbic acid or its derivative and a hydroquinone derivative in a purified water (pure water) such as distilled water, deionized water or the like, in the above-mentioned concentrations.

In FIG. 1, 6 is a pump disposed in the middle of a pipe 8 for feeding of aqueous electrolyte solution. By actuating this pump 6, the aqueous electrolyte solution 4 is sent to a diaphragm-free electrolytic cell 10 of continuous flow type via the pipe 8 for feeding of aqueous electrolyte solution. The flow rate of the aqueous electrolyte solution 4 fed to the diaphragm-free electrolytic cell 10 by the pump 6 is about 5 to 3,000 ml/min, preferably 50 to 1,000 ml/min, more preferably 500 to 1,000 ml/min.

The diaphragm-free electrolytic cell 10 has inside a pair of electrodes 12a and 12b facing each other. The pair of electrodes 12a and 12b are provided apart by a given distance. The distance is 2 to 0.05 mm, preferably 1.5 to 0.1 mm. When the distance is larger than 2 mm, the mixing of the anode side electrolytic water and the cathode side electrolytic water both generating by electrolysis is insufficient.

The electrodes 12a and 12b are made of an electrochemically inactive metal material. The electrode material is preferably platinum, a platinum alloy or the like.

16 is an electric source for electrolysis. Its plus terminal and minus terminal are connected to the electrodes 12a and 12b, respectively, by wirings 18 and 20. The polarities of the electric power applied to the electrodes are switched at given time intervals. By switching the polarities of the electric power applied at given time intervals, a cathode side electrolytic water and an anode side electrolytic water are produced at a pair of electrodes alternately; resultantly, the cathode side electrolytic water and the anode side electrolytic water are mixed efficiently. The time interval of polarity switch is preferably 2 to 1,200 times/min, more preferably 120 to 600 times/min. The switch of polarities can effectively prevent scale deposition on electrodes.

The aqueous electrolyte solution sent to the diaphragm-free electrolytic cell 10 via the pipe 8 for feeding of aqueous electrolyte solution is electrolyzed in the diaphragm-free electrolytic cell 10.

The hydroquinone derivative is not influenced by the electric field of electrolysis in a short time interval and shows substantially no migration caused by the electric field. The distribution of the electrolytic water produced is substantially constant in any portion of the aqueous electrolyte solution when sampling is made from each portion of the aqueous electrolyte solution.

The electric current in electrolysis is preferably 0.003 to 0.1 A/cm², more preferably 0.01 to 0.08 A/cm², particularly preferably 0.01 to 0.03 A/cm². When the electric current in electrolysis is less than 0.003 A/cm², it is impossible to make the amounts of dissolved oxygen and dissolved hydrogen in electrolytic water, higher than those in to-be-electrolyzed water. When the electric current in electrolysis is more than 0.1 A/cm², the oxidation of L-ascorbic acid proceeds at an accelerated rate, the amount of dehydroascorbic acid formed is large, and the possibility of formation of oxidized type L-ascorbic acid is high.

By the electrolysis mentioned above, the anode side electrolytic water and the cathode side electrolytic water both produced during electrolysis in the electrolytic cell are mixed with each other spontaneously. The mixed electrolytic water of the two electrolytic waters is taken outside continuously via a pipe 22 for taking-out of mixed electrolytic water.

Incidentally, there may be used, as the electrolytic cell 10, an electrolytic cell having a diaphragm between the two electrodes 12a and 12b. As the diaphragm, there can be appropriately used those conventionally used as an electrolytic diaphragm, such as ion exchange membrane, chargeless membrane and the like.

In the above description, the electrodes were one pair; however, a plurality of pairs may be provided in the electrolytic cell for higher electrolysis efficiency. Also, the polarities of electrodes need not be switched.

As to the electrolytic apparatus used in production of the electrolytic water of the present invention, there is no particular restriction, and any of electrolytic apparatuses conventionally used in production of electrolytic water may be used. Thus, there may be used any type regardless of the size of electrolytic apparatus, the use of diaphragm in electrolytic cell, etc.

In the above description, the electrolytic water was taken out as a mixed electrolytic water of cathode side electrolytic water and anode side electrolytic water. However, the cathode side electrolytic water and the anode side electrolytic water may be taken out separately and used separately. In this case, it is rational not to switch the polarities of electric power applied to the electrodes.

FIG. 2 is an other example of the electrolytic apparatus used in production of the electrolytic water of the present invention and is a schematic sectional view showing an electrolytic apparatus having a means for spraying an electrolytic water produced. In the electrolytic apparatus shown in FIG. 2, the left side of sheet is a front side of the apparatus and the right side of sheet is a back side of the apparatus.

In FIG. 2, 30 is an electrolytic apparatus, and 32 is a synthetic resin-made casing whose external shape is approximately cuboidal. Inside the casing 32 and at its top end, back side is formed a tank 34 for to-be-electrolyzed water.

At the top end of the tank 34 for to-be-electrolyzed water is fitted a feeding lid 36. By opening this lid 36, a feed solution (a to-be-electrolyzed water) can be fed into the tank 34 for to-be-electrolyzed water, from outside of the casing 32.

The tank 34 for to-be-electrolyzed water comprises a main portion 34a and a solution-feeding portion 34b projecting downward from the main portion 34a.

In the front side wall 38 of the solution-feeding portion 34b is formed a short-cylindrical, electrolytic chamber 40. In the back side wall 42 of the solution-feeding portion 34b is formed an open portion (a port 44 for plunger insertion).

Into the electrolytic chamber 40 are inserted one pair of rod-shaped electrodes 46, in parallel.

To the front end of the electrolytic chamber 40 is fitted a cylindrical, lined cap 48 which is formed so as to become narrower in diameter toward the front end. The open portion at the front end of the lined cap 48 is a feeding port 50. The feeding port 50 and its circumference are covered with a solution-absorbing medium 52 made of a foamed resin. The periphery of the solution-absorbing medium 52 is fixed to the wall of the electrolytic chamber 40 by a front cap 54. In front of the feeding port 50 is provided a perforated plate 56 via the solution-absorbing medium 52. In front of the feeding port 50 is also formed a spray port 58 on the casing 32.

The perforated plate 56 has a large number of very small holes of 18 to 24 μm diameter, and the top end thereof is fixed to a piezo trembler 60. The piezo trembler 60 vibrates when an alternate current or a pulse voltage is applied thereto, which vibrates the perforated plate 56 fixed to the piezo trembler 60.

Into the port 44 for plunger insertion is inserted a valve-driven plunger 62 of approximately cylindrical shape. The front end of the valve-driven plunger 62 is closed; and to the circumference of the front end is fitted a ring-shaped switching valve 64. At the back end (which is open) of the valve-driven plunger 62 is formed a flange 66. To the periphery of the switching valve 64 and the port 44 for plunger insertion, formed at the back side wall 42 is air-tightly fitted the two ends of a cylindrical diaphragm 65 formed in a pleated shape.

Inside the valve-driven plunger 62 is inserted a rod-shaped guide shaft 68 forward from the back side open end. The guide shaft 68 is formed in one piece with the casing 32 and its central axis agrees with the central axis of the valve-driven plunger 62. The valve-driven plunger 62 is fitted slidably along the axis direction of the guide shaft 68. As the valve-driven plunger 62 moves forward and backward along the guide shaft, the switching valve 64 moves forward and backward.

The valve-driven plunger 62 is pressed forward from backward by a compression coil spring 70 which surrounds the guide shaft 68. The valve-driven plunger 62 is located ordinarily in front of the guide shaft 68.

At the periphery of the switching valve 64 is formed a projected portion 64a which projects forward. When the projected portion 64a is pressed and hits the front side wall 38 of the solution-feeding portion 34b, the flow of to-be-electrolyzed water between the tank 34 for to-be-electrolyzed water and the electrolytic chamber 40 is prevented.

Between the back side wall 42 of the solution-feeding portion 34b and the flange 66 of the valve-driven plunger 62 is provided a rotary lever 74. The rotary lever 74 is fitted, at the bottom, to a horizontal plate 78 formed in one piece with the casing 32, by a pin 76. The top end of the rotary lever is engaged with the flange 66.

In FIG. 2, 80 is an actuator rod, 82 is an electric switch, 84 and 86 are each a wiring, and 88 is a push button. The wirings 84 and 86 are electrically connected to the electrodes 46 and the piezo trembler 60.

When the push button 88 provided in front of the electrolytic apparatus is pushed horizontally, the rotary level 74 is pushed and migrates backward while rotating. Owing to the backward migration (tilting) of the rotary lever 74, the flange 66 is pulled by the rotary lever 74, causing the backward migration of the valve-driven plunger 62 and the switching valve 64. Owing to the backward migration of the switching valve 64, a gap appears between the projected portion 64a and the front side wall 38, and the to-be-electrolyzed water in the tank 34 is fed into the electrolytic chamber 40.

When the push button 88 is pushed backward, the rotary lever 74 is migrated; simultaneously therewith, the electric switch 82 is pushed backward, which applies a voltage to the electrodes 46 and the piezo trembler 60.

The to-be-electrolyzed water fed into the electrolytic chamber 40 is electrolyzed by the voltage applied to the electrodes, whereby is produced a mixed electrolytic water of cathode electrolytic water and anode electrolytic water.

The mixed electrolytic water produced in the electrolytic chamber 40 is discharged from the feeding port 50, absorbed by the solution-absorbing medium, and is taken out from the electrolytic chamber 40. The mixed electrolytic water absorbed by the solution-absorbing medium 52 undergoes the vibration of the perforated plate 56 fixed to the piezo trembler 60, passes through the fine holes of the perforated plate 56, and is sprayed as fine droplets outside from the spray port 58.

Incidentally, in the electrolytic apparatus of FIG. 2, the distance between electrodes, the current density in electrolysis, the time interval of switch of polarities of the voltage applied to electrodes, the flow rate of to-be-electrolyzed water, etc. are the same as those in the electrolytic apparatus of FIG. 1.

### EXAMPLES

### Example 1

Using an electrolytic apparatus shown in FIG. 2, there was electrolyzed a to-be-electrolyzed water obtained by dissolving, in purified water, 34 mM of L-ascorbic acid, 0.136 mass % of α-arbutin and 0.5 mass t of citric acid, whereby an electrolytic water was produced. The electrolytic cell of the electrolytic apparatus was constituted by inserting two plate-shaped platinum electrodes of 20 mm² in effective electrode area (the distance between the electrodes: 0.85 mm) into a cylindrical electrolytic chamber 40 of 10 mm in inner diameter. The to-be-electrolyzed water was fed into the electrolytic cell at a flow rate of 10 ml/min and electrolyzed at a current density of 0.05 A/cm², and an electrolytic water was sprayed from a spray port 58. Incidentally, since a purified water was used as the solvent of electrolysis, the content of water-soluble inorganic salt in to-be-electrolyzed water was extremely small (less than 0.01 mM).

Using the electrolytic water obtained above, a sensory test was conducted for 8 females of twenties to fifties. The coating of electrolytic water on skin was conducted by spraying the electrolytic water taken directly from the electrolytic apparatus, to the front side of face. The times of spraying were two times per day (morning and evening) and the test duration was 2 weeks.

In the sensory test, semantic differentiation was used and skin condition was examined according to a method ordinarily conducted by testees. In the examination, “same” was 0 point; “slightly bad” was −1 point; “bad” was −2 point; “very bad” was −3 point; “slightly good” was +1 point; “good” was +2 point; and “very good” was +3 point. The results are shown in Table 1.

TABLE 1

| Evaluation items | Example 1 |
|---|---|
| Free from dryness (wet feeling) | +5 |
| Smoothness (smooth feeling) | +5 |
| Free from fatty feeling (sticky feeling) | +5 |
| Firmness | +6 |
| Luster | +9 |
| Fineness of pores and texture | +4 |
| Prevention and amelioration of pimples and | 0 |
| eruptions | |
| Free from dark spot | +7 |
| Amelioration of blotches and freckles | +1 |
| Free from reddish tinge | +1 |
| Makeup | +5 |
| Wearing of makeup (no coming-off of makeup) | +3 |
| Overall condition | +9 |

The electrolytic water of the present invention was superior in amelioration of dark spots (which is a skin-whitening effect caused by α-arbutin) as well as in firmness, luster, smoothness, no sticky feeling, etc. of skin.

The electrolytic water obtained was measured for pH, oxidation and reduction potential (ORP), dissolved oxygen amount (DO) and electrical conductance (EC). The results are shown in Table 2.

TABLE 2

| | | ORP | DO | EC |
|---|---|---|---|---|
| | pH | (mV) | (mg/L) | (mS/m) |
| To-be-electrolyzed water | 2.42 | 152.3 | 3.06 | 66.2 |
| Electrolytic water of Example 1 | 2.55 | −83.2 | 3.12 | 66.2 |

What is claimed is:

1. An electrolytic water containing less than 0.1 mM of a water-soluble inorganic salt, 0.05 to 0.5 mass t of a hydroquinone derivative and 1 to 50 mM of L-ascorbic acid or a derivative thereof.

2. An electrolytic water according to claim 1, wherein the L-ascorbic acid derivative is sodium ascorbyl phosphate and/or magnesium ascorbyl phosphate.

3. An electrolytic water according to claim 1, wherein the hydroquinone derivative is α-arbutin or 6-arbutin.

4. A process for producing an electrolytic water, which comprises electrolyzing a to-be-electrolyzed water containing less than 0.1 mM of a water-soluble inorganic salt, 0.05 to 0.5 mass % of a hydroquinone derivative and 1 to 50 mM of L-ascorbic acid or a derivative thereof and taking out the electrolytic water obtained.

5. A process for producing an electrolytic water according to claim 4, wherein the L-ascorbic acid derivative is sodium ascorbyl phosphate and/or magnesium ascorbyl phosphate.

6. A process for producing an electrolytic water according to claim 4, wherein the hydroquinone derivative is α-arbutin or β-arbutin.

7. A process for producing an electrolytic water according to claim 4, wherein the electrolysis is conducted at a current density of 0.003 to 0.1 A/cm².

8. A process for producing an electrolytic water according to claim 4, wherein the to-be-electrolyzed water containing less than 0.1 mM of a water-soluble inorganic salt, 0.05 to 0.5 mass % of a hydroquinone derivative and 1 to 50 mM of L-ascorbic acid or a derivative thereof is fed into a diaphragm-free electrolytic cell of continuous flow type at a flow rate of 5 to 3,000 l/min and the electrolysis is conducted continuously at a current density of 0.003 to 0.1 A/cm².
